(19) United States
(12) Patent Application Publication
(10) Pub. No.: US 2024/0197856 A1
(43) Pub. Date: Jun. 20, 2024

(54) IMMUNOGENIC COMPOSITION AND USES THEREOF

(71) Applicant: MediDiamond Inc., Taipei City (TW)

(72) Inventors: Hsin-Hung LIN, Taipei City (TW); Long-Jyun SU, Taipei City (TW); Yi-Chia WU, Taipei City (TW)

(21) Appl. No.: 18/535,827

(22) Filed: Dec. 11, 2023

Related U.S. Application Data
(60) Provisional application No. 63/432,566, filed on Dec. 14, 2022.

(51) Int. Cl. A61K 39/145; A61K 47/52; A61K 47/64; A61P 31/16; C07K 14/11; C12N 7/00

(52) U.S. Cl. CPC A61K 39/145; A61K 47/52; A61K 47/646; A61P 31/16; C07K 14/11; C12N 7/00; B82Y 5/00

(57) ABSTRACT

Disclosed herein is an immunogenic composition for enhancing immunogenicity and treatment efficacy. The immunogenic composition comprises a nanoparticle (e.g., a nanodiamond) and an immunogenic conjugate having formula (I): X-L-Y, wherein X is an agonist module, L is a linker unit, and Y is an antigen. In the immunogenic composition, the immunogenic conjugate is coupled to the outer surface of the nanoparticle. Also encompasses herein is a method of preventing or treating a disease in a subject in need thereof, comprising administering an effective amount of the immunogenic composition set forth above to the subject.

### CROSS-REFERENCES TO RELATED APPLICATIONS

This application claims priority and the benefit of U.S. Provisional Patent Application No. 63/432,566, filed Dec. 14, 2022, the entirety of which is incorporated herein by reference.

### BACKGROUND OF THE INVENTION

### 1. Field of the Invention

The present disclosure relates to immunogenic composition and uses thereof. More particularly, the disclosed invention relates to a novel immunogenic composition comprising a nanodiamond and an immunogenic conjugate coupled thereto, as well as a method of preventing and/or treating diseases with the aid of the immunogenic composition.

### 2. Description of Related Art

Vaccine trains the adaptive immune system to either generate immunological memory before infection or to recognize ongoing diseases, thus, vaccination is one of the most successful biomedical interventions for both prophylactic and therapeutic treatments. In over two centuries, prophylactic vaccines against fatal infections have reached extraordinary successes such as complete eradication of smallpox, anthrax, and plaque. Recent advances in therapeutic vaccines provide solutions for treating incurable illnesses, such as cancers and human immunodeficiency virus (HIV) infection. Despite the successes of conventional vaccines so far, there exist inadequacies, thus, improvements in eliciting a satisfactory level of immunity, eliminating instability and toxicity in vivo, and increasing stability for multiple-dose immunizations, are still required.

To increase the efficacy of vaccination, adjuvants act as stimulators and are typically added to vaccines to enhance the magnitude and durability of the immune response. Recently, nanoparticles (NPs) have been reported to work well in oil emulsion, evoking sustained antibody responses and T cell activation, and thus may serve as a potent vaccine adjuvant. However, the development of vaccines containing novel adjuvants is reported as the slowest process in medical research and, therefore failed to be in line with industrial needs, particularly when facing an emerging disease, e.g., the recent COVID-19 pandemic in 2019.

To this end, a vaccine platform is proposed and developed. Formed by combining various tailorable modules including essential elements (e.g., antigens and adjuvants), the vaccine platform allows researchers to design suitable antigens and adjuvants based on different practical strategies, thereby accelerating the vaccine development. In this respect, how to successfully combine and synergize adjuvants and antigens in the platform becomes an issue to be addressed.

Given the foregoing, there exists in the related art a need for an improved platform for modularizing the entire bioprocess for enhancing immunogenicity.

### SUMMARY

The following presents a simplified summary of the disclosure to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description presented later.

As embodied and broadly described herein, one aspect of the present disclosure is directed to an immunogenic composition comprising a nanoparticle and an immunogenic conjugate of formula (I): X-L-Y. In the formula (I), X is an agonist module, L is a linker unit, and Y is an antigen. In immunogenic composition, the immunogenic conjugate is coupled to the outer surface of the nanoparticle. Typically, the nanoparticle may be made of inorganic-based, carbon-based, and organic-based materials such as metals, metal oxides, graphene, carbon fibers, dendrimers, cyclodextrin, liposomes, and micelles. According to some embodiments of the present disclosure, the nanoparticle is made of diamond particles, that is, a nanodiamond.

According to some embodiments of the present disclosure, the nanodiamond may be about 1 to 1,000 nm in diameter.

According to some embodiments of the present disclosure, the agonist module may be an agonist selected from the group consisting of a Toll-like receptor (TLR) agonist, a retinoic acid-inducible gene I (RIG-I)-like receptor agonist, a C-type lectin receptor (CLR) agonist, a NOD-like receptor (NLR) agonist, a stimulator of interferon genes (STING) agonist, and a pattern recognition receptor (PRR) agonist.

Example of the TLR agonist suitable for use in the present immunogenic conjugate includes, but is not limited to, a TLR5 agonist, a TLR7 agonist, and a TLR9 agonist. In one working embodiment, the TLR5 agonist is flagellin. In another working embodiment, the TLR7 agonist is 1H-imidazo[4,5-C]quinoline-1-propanamine,4-amino-2-(ethoxymethyl). In still another working embodiment, the TLR9 agonist is cytosine-phosphate-guanine oligonucleotide (ODN) 2006 having a substituted amino group at its 5′-end (ODN2006-NH₂).

According to some embodiments of the present disclosure, the linker unit is a succinimide derivative or a PEG-based compound. Example of the succinimide derivative suitable for use as the linker unit in the present disclosure includes but is not limited to N-(ε-maleimidocaproyloxy) succinimide ester or N-γ-maleimidobutyryl-oxysuccinimide ester. Example of the PEG based compound suitable for use as the linker unit in the present disclosure includes but is not limited to Azido-PEG4-NHS ester (NHS-PEG4-Azide).

According to some embodiments of the present disclosure, the antigen is derived from an autogenous, allogeneic, heterophile, or xenogeneic source.

In some working embodiments, the antigen is an autoantigen.

Examples of the autoantigen suitable for use in the present immunogenic composition include those derived from a protein that includes but is not limited to, proinsulin, myelin basic protein (MBP), insulin, aluminum-formulated glutamic acid decarboxylase (GAD-alum), amyloid precursor protein (APP), heat shock protein, major histocompatibility complex (MHC), human leukocyte antigen (HLA), myelin oligodendrocyte glycoprotein (MOG), and ovalbumin.

According to some embodiments of the present disclosure, the antigen is a tumor antigen, a bacterial antigen, a viral antigen, a fungal antigen, a parasitic antigen, or a combination thereof.

Examples of the tumor antigen suitable for use in the present immunogenic composition include, but are not limited to, neoantigen, tumor-derived lysate, alpha-fetoprotein (AFP), carcinoembryonic antigen (CEA), mucin, epithelial tumor antigen (ETA), tyrosinase, melanoma-associated antigen (MAGE), RAS protein, and tumor suppressor protein.

Examples of the bacterial antigen suitable for use in the present immunogenic composition include those derived from a bacterial species, including but not limited to, genera of Actinomyces, Aeromonas, Arthrobacter, Bacillus, Bacteroides, Bordetella, Borrelia, Brucella, Campylobacter, Chlamydia, Citrobacter, Clostridium, Corynebacterium, Escherichia, Enterobacter, Gardnerella, Helicobacter, Haemophilus, Klebsiella, Legionella, Listeria, Mycobacterium, Neisseria, Nocardia, Pasteurella, Proteus, Pseudomonas, Ureaplasma, Salmonella, Shigella, Spirillum, Spirochaeta, Staphylococcus, Streptobacillus, Streptococcus, Streptomyces, Treponema, and Yersinia.

Examples of the viral antigen suitable for use in the present immunogenic composition include those derived from a viral species, which belongs to but is not limited to, genera of Adenovirus, Alphacoronavirus, Betacoronavirus, Cytomegalovirus, Deltainfluenzavirus, Deltacoronavirus, Gammacoronavirus, Hepatovirus, Influenza A virus, Influenza B virus, Influenza C virus, Influenza D virus, Lentivirus, Letovirus, Lymphocryptovirus, Orthopneumovirus, Orthopoxvirus, Papillomavirus, Quaranjavirus, Rotavirus, Simplexvirus, and Varicellovirus. In one working embodiment, the viral antigen is a hemagglutinin of the Influenza A virus.

Example of the fungal antigen is derived from a fungal species that causes a fungal infection, which includes but is not limited to aspergillosis, blastomycosis, candidiasis, chromoblastomycosis, cryptococcosis, histoplasmosis, mycetoma, paracoccidioidomycosis, ringworm, and tinea versicolor.

Example of the parasitic antigen is derived from a parasite species that causes a parasitic infection, which includes, but is not limited to African trypanosomiasis, amebiasis, Chagas disease, echinococcosis, fascioliasis, hookworm disease, hymenolepis, leishmaniasis, neurocysticercosis, onchocerciasis, Plasmodium infection, paragonimiasis, Pneumocystis pneumonia (PCP), schistosomiasis, trichomoniasis, taeniasis, and trichuriasis.

Another aspect of the present disclosure is directed to a method for preventing or treating a disease in a subject in need thereof, comprising administering an effective amount of the present immunogenic composition set forth above to the subject.

According to some embodiment of the present disclosure, the disease is a cancer or an infectious disease.

Examples of the cancer treatable by the present method include, but are not limited to, bladder cancer, bone cancer, bone marrow cancer, brain cancer, breast cancer, cholangiocarcinoma, colon cancer, esophagus cancer, gastrointestinal cancer, gum cancer, head and neck cancer, kidney cancer, liver cancer, lung cancer, nasopharyngeal carcinoma, leukemia, lymphoma, ovary cancer, prostate cancer, skin cancer, stomach cancer, testis cancer, tongue cancer, and uterus cancer.

Examples of the infectious disease treatable or preventable by the present method are caused by a pathogen, which includes, but is not limited to, bacterium, virus, fungus, and parasite. In some embodiments, the infectious disease is caused by Influenza A virus, Influenza B virus, Influenza C virus, Influenza D virus, severe acute respiratory syndrome-related coronavirus (SARS-CoV-2), or a combination thereof.

According to some embodiments of the present disclosure, the subject is a mammal, preferably a human.

Many of the attendant features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawing, where:

FIG. 1 is a timeline depicting an experimental design for single-dose vaccinating mice with the present immunogenic compositions: “HA-TLR7-LVA” and “HA-TLR9-LVA” in accordance with one embodiment of the present disclosure;

FIG. 2 are bar graphs respectively depicting the quantitative results of the titers of IgG (panel A) and its three subclasses: IgG1 (panel B), IgG2a (panel C), and IgG2b (panel D) after immunization of the present HA-TLR7-LVA in accordance with the time line depicted in FIG. 1;

FIG. 3 are bar graphs respectively depicting the quantitative results of the titers of IgG (panel A) and its three subclasses: IgG1 (panel B), IgG2a (panel C), and IgG2b (panel D) after immunization of the present HA-TLR9-LVA in accordance with the time line depicted in FIG. 1;

FIG. 4 is a timeline depicting an experimental design for double-vaccine immunizing mice with the present immunogenic compositions “HA-TLR5-LVA” and “HA-TLR7-LVA” in accordance with one embodiment of the present disclosure;

FIG. 5 are bar graphs respectively depicting the quantitative results of the titers of IgG (panel A) and its three subclasses: IgG1 (panel B), IgG2a (panel C), and IgG2b (panel D) after double immunization of the present HA-TLR5-LVA in accordance with the timeline depicted in FIG. 4;

FIG. 6 are bar graphs respectively depicting the quantitative results of intracellular cytokines of T cells: IFN-γ (panel A), IL-2 (panel B), TNF-α (panel C), CD40L (panel D), and CD107a (panel E) after double immunization of the present HA-TLR5-LVA in accordance with the timeline depicted in FIG. 4;

FIG. 7 are bar graphs respectively depicting the quantitative results of intracellular cytokines of T cells: IFN-7 (panel A), IL-2 (panel B), TNF-α (panel C), and CD40L (panel D) after double immunization of the present HA-TLR7-LVA in accordance with the timeline depicted in FIG. 4

FIG. 8 is a timeline depicting an experimental design for double-vaccine immunizing mice with the present immunogenic compositions “HA-TLR7-LVA” and “HA-TLR9-LVA” in accordance with one embodiment of the present disclosure;

FIG. 9 are bar graphs respectively depicting the quantitative results of the titers of IgG (panel A) and its three subclasses: IgG1 (panel B), IgG2a (panel C), and IgG2b (panel D) after double immunization of the present HA-TLR7-LVA in accordance with the timeline depicted in FIG. 8;

FIG. 10 are bar graphs respectively depicting the quantitative results of intracellular cytokines of T cells: IFN-7 (panel A), IL-2 (panel B), TNF-α (panel C), CD40L (panel D), and CD107a (panel E) after double immunization of the present HA-TLR7-LVA in accordance with the timeline depicted in FIG. 8;

FIG. 11 are bar graphs respectively depicting the quantitative results of the titers of IgG (panel A) and its three subclasses: IgG1 (panel B), IgG2a (panel C), and IgG2b (panel D) after double immunization of the present HA-TLR9-LVA in accordance with the timeline depicted in FIG. 8; and

FIG. 12 are bar graphs respectively depicting the quantitative results of intracellular cytokines of T cells: IFN-7 (panel A), IL-2 (panel B), TNF-α (panel C), and CD107a (panel D) after double immunization of the present HA-TLR9-LVA in accordance with the timeline depicted in FIG. 8.

### DESCRIPTION

The detailed description provided below in connection with the appended drawing is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definitions

For convenience, certain terms employed in the specification, examples, and appended claims are collected here. Unless defined otherwise, all technical and scientific terms used herein have the same meaning as commonly understood by one of the ordinary skills in the art to which this invention belongs.

The singular forms “a”, “and”, and “the” are used herein to include plural referents unless the context clearly dictates otherwise.

The term “agonist module” used herein refers to a substance capable of inducing or enhancing signal transduction resulting from antigen-binding at the cell level, thereby synergistically working with the antigen to enhance the immune response in vivo. In the present disclosure, the agonist module is coupled to an antigen via a linker unit thereby forming a conjugate, and the thus produced immunogenic conjugate may serve as a tailored platform adapting to various targets or conditions. According to embodiments of the present disclosure, the agonist module is an agonist.

The terms “autogenous” or “autologous” are interchangeably used herein to encompass the biomaterials (e.g., genetic materials, peptides, antigens, and immune cells) that originate or are derived from sources within the same individual. Examples of the autogenous antigens include but are not limited to, autoantigens.

The term “allogeneic” peptides or antigens is used herein to refer to antigenic fragments including polypeptides or polynucleotides encoding the polypeptides that originated from different individuals of a given species. Examples of the allogeneic antigens include but are not limited to, human major histocompatibility complex (MHC) molecules and human blood antigens among different individuals.

The term “heterophile antigen(s)”, “heterophil antigen(s)” or “heterogenetic antigen(s)” are used interchangeably herein to encompass antigens of similar nature that are present in different tissues across different biological species, and capable of stimulating the production of antibodies that cross-react with different tissues from other organisms, which are mostly animals.

Examples of the heterophile antigens include Forssman antigen that is widely present in Carnivora and Artiodactyla; antigens of OX (OX 19, OX 2, and OXK) strains of Proteus species used to make a diagnosis of rickettsial infections; and viral capsid antigen (VCA) for detecting infectious mononucleosis caused by Epstein-Barr virus (EBV); but are not limited thereto.

The term “a xenogeneic antigen” as used herein to encompass antigens derived from a different species.

The term “tumor antigen” as used herein is intended to encompass the antigens that are directly or indirectly related to tumor cells, and capable of triggering an immune response in a host. Tumor antigens are broadly classified into two categories: (1) tumor-specific antigens (TSA), which are present only on tumor cells; and (2) tumor-associated antigens (TAA), which are present on both tumor and normal cells while having an expression difference therebetween. Examples of the tumor antigens include neoantigens, tumor-derived lysates, alpha-fetoprotein (AFP), carcinoembryonic antigen (CEA), mucin proteins, an epithelial tumor antigen (ETA), tyrosinase, a melanoma-associated antigen (MAGE), a RAS protein, and tumor suppressor proteins.

The term “neoantigen” as used herein refers to one type of TSA that is encoded by somatic mutations present in cancer driver genes, or by private mutations usually found in a single-family or small population.

The term “treating” or “treatment” as used herein is intended to mean obtaining a desired pharmacological and/or physiologic effect, e.g., reducing the proliferation of tumor or cancer cells, and/or inhibiting the progression of metastatic cancers in a subject. The effect may be prophylactic in terms of completely or partially preventing a symptom thereof and/or therapeutic in terms of a partial or complete cure for a disease and/or adverse effects attributable to the disease. “Treatment” as used herein includes curative or palliative treatment of a disease in a mammal, particularly human; and includes: (1) inhibiting a condition (e.g., by arresting a cancer's progression in a subject, or by eliminating pathogens in the subject); or (2) relieving a condition (e.g., inhibiting the tumor metastasis in the subject).

The terms “subject” or “patient” are used interchangeably herein to mean a mammal including the human species that is treatable by the method of the present disclosure. The term “mammal” refers to all members of the class Mammalia, including humans, primates, domestic and farm animals, such as rabbits, pigs, sheep, and cattle; as well as zoo, sports, or pet animals; and rodents, such as mice and rats. Further, the term “subject” or “patient” is intended to refer to both the male and female gender unless one gender is specifically indicated.

### 2. Detail Description of Preferred Embodiments

Nanodiamonds (NDs) are carbon-based nanomaterials (i.e., diamond) with a diameter of less than 1 mm and can be modified with functional proteins, such as fluorescent proteins, while maintaining extraordinary chemical stability and biological inertness. It is also reported that NDs-in-oil emulsion formulation is capable of increasing antibody titer and enhancing T cell activation. The present disclosure is based, at least in part, on the discovery that nanodiamond adsorbs proteins onto its outer surface and, therefore may be used as a desired material for developing a vaccine platform that facilitates the efficiency of vaccination. Accordingly, the present disclosure aims to provide an immunogenic composition comprising tailorable modules for enhancing immunogenicity and treatment efficacy.

### 2.1 Immunogenic Compositions of the Present Disclosure

One aspect of the present disclosure is directed to a novel immunogenic composition, which comprises a nanodiamond and an immunogenic conjugate coupled to the outer surface of the nanodiamond. The immunogenic conjugate is of a formula (I): X-L-Y, wherein X is an agonist module, L is a linker unit, and Y is an antigen.

According to some embodiments of the present disclosure, the present immunogenic composition is prepared by mixing an emulsion of nanodiamonds with the immunogenic conjugates via continuous shaking, such that the immunogenic conjugates are aggregated and adsorbed onto the outer surface of each nanodiamond, thereby producing the present immunogenic composition. In some embodiments, the immunogenic conjugate having been adsorbed onto the outer surface of nanodiamond has a configuration in which the antigen is proximal to the nanodiamond, and the agonist module is distal to the nanodiamond. In some preferred embodiments, the immunogenic conjugate is adsorbed onto the outer surface of nanodiamond via a hydrophobic interaction between the antigen and the nanodiamond.

In some embodiment, the nanodiamond suitable for use in the present disclosure has a diameter ranging from 1 to 1,000 nm, such as 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260,270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 nm; more preferably, each nanodiamond is about 100 nm in diameter.

According to some embodiments of the present disclosure, in the immunogenic conjugate, the agonist module is an agonist that activates a receptor to elicit a biological response. According to embodiments of the present disclosure, when being coupled to the nanodiamond, the agonist of the immunogenic conjugate can enhance or induce the immune response in living subjects.

Example of the agonist suitable for use in the present immunogenic composition includes but is not limited to, a Toll-like receptor (TLR) agonist, a retinoic acid-inducible gene I (RIG-I)-like receptor agonist, a C-type lectin receptor (CLR) agonist, a NOD-like receptor (NLR) agonist, a stimulator of interferon genes (STING) agonist, a pattern recognition receptor (PRR) agonist, and a combination thereof.

In preferred embodiments, the agonist is the TLR agonist including but not limited to, TLR5 agonists, TLR7 agonists, and TLR9 agonists. In one working example, when the TLR agonist is a TLR5 agonist, the agonist is flagellin. In another working example, the TLR7 agonist, 1H-imidazo[4,5-C]quinoline-1-propanamine,4-amino-2-(ethoxymethyl), is coupled to the linker unit. In still another working example, the TLR9 agonist is cytosine-phosphate-guanine oligonucleotide (ODN) 2006 having a substituted amino group at its 5′-end (ODN2006-NH₂).

According to some embodiments of the present disclosure, the linker unit of the immunogenic conjugate is a molecule having carbonyl groups that may react with amine groups of another molecule thereby forming amide bonds therebetween. Preferred examples of the linker unit include but are not limited to succinimide derivatives, such as N-(ε-maleimidocaproyloxy) succinimide ester (EMCS), N-γ-maleimidobutyryl-oxysuccinimide ester (GMBS) and the like. According to alternative embodiments, the linker unit of the immunogenic conjugate is a PEG-based compound having an azido group at one end and an N-hydroxysuccinimide (NHS) group at another end, in which the azido group may undergo nucleophilic substitution or Michael-like conjugate addition, while the NHS group may react with amine group to form an amide linkage therebetween. Example of the PEG-based compound suitable for use as the linker unit includes but is not limited to Azido-PEG4-NHS ester (NHS-PEG4-Azide), and Azido-PEG12-NHS ester. In one working example, the linker unit in the present immunogenic conjugate is Azido-PEG4-NHS ester.

According to some embodiments of the present disclosure, antigen capable of triggering immune response (i.e., stimuli of T cell activation in vivo) may be an antigenic polypeptide or a polynucleotide encoding the antigenic polypeptide derived from an autogenous, allogeneic, heterophile or xenogeneic source.

In some embodiments, the antigen used in the present immunogenic composition may be derived from an autoantigen and is recognized by the immune system of subjects suffering from a specific autoimmune disease. Examples of the autoantigen suitable for use in the present immunogenic composition may be those derived from a protein, which includes but is not limited to, proinsulin, myelin basic protein (MBP), insulin, aluminum-formulated glutamic acid decarboxylase (GAD-alum), amyloid precursor protein (APP), heat shock protein, major histocompatibility complex (MHC), human leukocyte antigen (HLA), myelin oligodendrocyte glycoprotein (MOG), ovalbumin, and a combination thereof.

According to alternative or optional embodiments of the present disclosure, the antigen of the immunogenic conjugate may be a tumor antigen, a fungal antigen, a parasitic antigen, a bacterial antigen, a viral antigen, or a combination thereof.

Examples of the tumor antigens suitable for use in the present immunogenic composition include but are not limited to, neoantigen, tumor-derived lysate, alpha-fetoprotein (AFP), carcinoembryonic antigen (CEA), mucin, epithelial tumor antigen (ETA), tyrosinase, melanoma-associated antigen (MAGE), RAS protein, tumor suppressor protein, and a combination thereof.

Examples of the fungal antigens suitable for use in the present immunogenic composition include those derived from a fungal species that causes a fungal infection, which includes but is not limited to, aspergillosis, blastomycosis, candidiasis, chromoblastomycosis, cryptococcosis, histoplasmosis, mycetoma, paracoccidioidomycosis, ringworm, tinea versicolor, and a combination thereof.

Examples of the parasitic antigens suitable for use in the present immunogenic composition include those derived from a parasite species that causes a parasitic infection including but not limited to, African trypanosomiasis, amebiasis, Chagas disease, echinococcosis, fascioliasis, hookworm disease, hymenolepis, leishmaniasis, neurocysticercosis, onchocerciasis, Plasmodium infection, paragonimiasis, Pneumocystis pneumonia (PCP), schistosomiasis, trichomoniasis, taeniasis, trichuriasis, and a combination thereof.

Examples of the bacterial antigens suitable for use in the present immunogenic composition include those derived from a bacterial species, which includes genera of Actinomyces, Aeromonas, Arthrobacter, Bacillus, Bacteroides, Bordetella, Borrelia, Brucella, Campylobacter, Chlamydia, Citrobacter, Clostridium, Corynebacterium, Escherichia, Enterobacter, Gardnerella, Helicobacter, Haemophilus, Klebsiella, Legionella, Listeria, Mycobacterium, Neisseria, Nocardia, Pasteurella, Proteus, Pseudomonas, Ureaplasma, Salmonella, Shigella, Spirillum, Spirochaeta, Staphylococcus, Streptobacillus, Streptococcus, Streptomyces, Treponema, and Yersinia, but not limited thereto.

Examples of the viral antigens suitable for use in the present immunogenic composition include those derived from a viral species, which include but are not limited to, Adenovirus, Alphacoronavirus, Betacoronavirus, Cytomegalovirus, Deltainfluenzavirus, Deltacoronavirus, Gammacoronavirus, Hepatovirus, Influenza A virus, Influenza B virus, Influenza C virus, Influenza D virus, Lentivirus, Letovirus, Lymphocryptovirus, Orthopneumovirus, Orthopoxvirus, Papillomavirus, Quaranjavirus, Rotavirus, Simplexvirus, and Varicellovirus. In working examples, the viral antigen is a hemagglutinin of the Influenza A virus.

By virtue of the above features, the present immunogenic composition possesses tailored modules of antigens and agonists that adapt to various target diseases, thus facilitating the development of vaccines.

### 2.2 Methods of Use

The present immunogenic composition is useful for enhancing immune responses in living subjects; thus, it may be used in the treatment or prophylaxis of diseases. The present disclosure thus encompasses a method for preventing or treating a disease in a subject in need thereof. The method mainly includes administering an effective amount of the afore-described immunogenic composition to the subject.

The present immunogenic composition may be formulated into solutions or suspensions, and such formulations can be administered parenterally to appropriate or desired sites of action and lesions (e.g., the tumor) of the subject. Exemplary suitable parenteral route includes but is not limited to, transdermal, percutaneous, intravenous, intramuscular, intra-arterial, intramedullary, intrathecal, intraventricular, intradermal, subcutaneous, rectal, intravaginal, and intraperitoneal routes. Suitable routes vary, as recognized by those skilled in the art, depending on the particular condition being treated, the severity of the condition, the individual patient parameters including age, physical condition, size, gender and weight, the duration of the treatment, the nature of concurrent therapy (if any), the dosage and the nature of active ingredients, genetic factors and like factors within the knowledge and expertise of the health practitioner. These factors are well known to those with ordinary skills in the art and can be addressed with no more than routine experimentation. In general, the most appropriate route of administration depends on a variety of factors including the agent's stability in the environment of the circulatory system, and/or the condition of the subject (e.g., the severity of lymphoma of the subject, or whether the subject is able to tolerate conventional treatment). According to some embodiments of the present disclosure, the present immunogenic composition is formulated into injectable formulation for subcutaneous administration.

The present immunogenic composition can be administered at a frequency that effectively prevents, inhibits, suppresses, or treats diseases, conditions, or traits in the subject. In some embodiments, the immunogenic composition can be administered once, or at a frequency of four times a day to once every three months; for example, at a frequency of four times a day, three times a day, twice a day, once a day, once every other day, once every third day, once every week, once every other week, once monthly, twice monthly, thrice monthly, once every other month, or once every three months. Preferably, the immunogenic composition is administered to the subject at a frequency of once per two weeks (once every fourteen days). Optionally, the immunogenic composition is administered to the subject once and for all.

According to embodiments of the present disclosure, diseases treatable by the present method may be cancers, and/or infectious diseases, and the like.

Examples of the cancer treatable by the present method include but are not limited to, bladder cancer, bone cancer, bone marrow cancer, brain cancer, breast cancer, cholangiocarcinoma, colon cancer, esophagus cancer, gastrointestinal cancer, gum cancer, head and neck cancer, kidney cancer, liver cancer, lung cancer, nasopharyngeal carcinoma, leukemia, lymphoma, ovary cancer, prostate cancer, skin cancer, stomach cancer, testis cancer, tongue cancer, and uterus cancer.

Alternatively, the infectious diseases treatable by the present method may be caused by a pathogen selected from the group consisting of a bacterium, a virus, a fungus, and a parasite. In some embodiments of the present disclosure, the infectious disease may be caused by the Influenza A virus, Influenza B virus, Influenza C virus, Influenza D virus, or severe acute respiratory syndrome-related coronavirus (SARS-CoV-2).

According to the present disclosure, the subject treatable by the present method includes humans, primates, domestic and farm animals, such as rabbits, pigs, sheep, and cattle; as well as zoo, sports, or pet animals; and rodents, such as mice and rats. In one working example, the subject is a mouse; in another working example, the subject is a human.

By virtue of the above features, the present method provides effective immunization for the subject, thereby allowing diseases (e.g., cancer and infectious disease) to be efficiently treated.

### EXAMPLES

### Materials and Methods

### Nanodiamonds (NDs)

Nanodiamonds (NDs) were prepared in the form of a synthetic monocrystalline powder with a homogeneous diameter of 100 nm (LTK1000-NF, LuminX Biotech). To remove metallic impurities and graphitic carbon atoms on the surface, the diamond powders were subjected to oxidization and electromagnetic radiation to functionalize their surfaces with —COOH groups for further conjugation with peptides or proteins.

### Protein (Antigen) Preparation

The cDNA sequence corresponding to hemagglutinin (HA) derived from A/California/07/2009 H1N1 virus (Accession No. ACP41953.1) with the C-terminal His-tag was constructed into pcDNA 3.4 vector (A14697, Invitrogen) for protein expression. The construct was transfected into Expi293F cells (A14527, Gibco) using the ExpiFectamine 293 transfection kit (A14525, Gibco), to produce the HA protein. The culture supernatants were collected and subjected to Ni²⁺-covalent-bound HisTrap excel prepacked column (17-3712-06, GE Healthcare) for protein purification, thus, the purified HA protein is obtained and collected.

### Immunogenic Conjugates Preparation

### I. Modification of Toll-Like Receptor (TLR) 7 and TLR9 Agonists

The TLR7 agonist used in the present examples was produced by modifying 1-[4-amino-2-(ethoxymethyl)imidazo[4,5-c]quinolin-1-yl]-2-methylpropan-2-ol (Resiquimod; R848) with a 5′-end amino modifier, thereby forming a TLR7 agonist of R848-NH₂, of which the chemical name is 1H-imidazo[4,5-C]quinoline-1-propanamine,4-amino-2-(ethoxymethyl).

The TLR9 agonist used in the present examples was produced by modifying cytosine-phosphate-guanine (CpG) oligonucleotide (ODN) 2006 (Agatolimod) with a 5′-end amino modifier, thereby forming the present TLR9 agonist, ODN 2006-NH₂.

### II. Agonist-Linker-Antigen

The present immunogenic conjugates were prepared by mixing the present TLR5 agonist (flagellin), TLR7 agonist (R848-NH₂), or TLR9 agonist (ODN 2006-NH₂) dissolved in 20 μL of ddH₂O with N,N-Diisopropylethylamine (DIPEA, 3.48 μL) and linker compounds (e.g., N-(ε-maleimidocaproyloxy) succinimide ester (EMCS) or N-γ-maleimidobutyryl-oxysuccinimide ester (GMBS)) dissolved in dimethyl sulfoxide (DMSO, 100 mM, 10 μL) in the DMSO solution with a total volume of 100 μL. The mixture was stirred at room temperature for 2 hours, until the crude form of agonist-linker conjugates was formed. The mixture was then subjected to centrifugation at 16000×g for 10 minutes to remove particulate matters, and the crude agonist-linker (or carrier protein-linker) is further purified by using reversed-phase HPLC (Atlantis T3, 5 m, 4.6×250 mm, C18 column) with an aid of TEAA/CAN (0.1 M, pH7.0) system. The fractions containing pure agonist-linker conjugates were thus collected and concentrated via evaporation and lyophilization, thereby obtaining a white solid for further use.

To reduce antigens, tris(2-carboxyethyl)phosphine (TCEP) solution (100 mM in ddH₂O, 13.7 μL) was mixed with a stock solution (400 μL, containing 50 mM 4-(2-hydroxyethyl)-1-piperazineethanesulfonic acid (HEPES) and 150 mM NaCl, pH7.2) of antigens (10 mg/mL, i.e., hemagglutinin (HA) of H1N1 virus) at a 50 molar equivalents of TCEP to the antigen. The mixture was reacted at room temperature for 1 hour. The reduced antigenic proteins are buffer-exchanged into a phosphate-buffered saline (PBS) solution (pH7.2, containing 10 mM EDTA) by using a desalting column (HiTrap desalting, 5 mL). The fractions containing antigens were thus collected and concentrated by ultrafiltration (Amicon 50 kDa), and subjected to a colorimetric analysis with 5,5′-dithiobis(2-nitrobenzoic acid) to confirm existence of free thiol groups on each antigen molecules. After confirmation, the agonist-linker conjugates dissolved in ultrapure water were added to the solution of antigens at various molar equivalents (5 to 15), and the reactions were reacted at room temperature overnight until the present conjugates of agonist-linker-antigen were formed. The mixture was then subjected to size-exclusion chromatography (Superdex 200 increase 5/150 GL, 8.6 μm, 5×153 mm, 3 mL) and anion exchange chromatography (Protein-pak Hi Res Q, 5 μm, 4.6×100 mm, 0 to 2 M NaCl gradient) to remove unconjugated antigens and agonists, so as to obtain the present immunogenic conjugate (i.e., the agonist-linker-antigen conjugate).

### Immunogenic Composition Preparation

The present immunogenic composition was prepared by mixing 5 μL solution of immunogenic conjugates (the concentration of 1-2 mg/mL) with NDs (30 μL NDs suspension at the dose 2 mg/mL) in a PBS solution at room temperature, and the mixture was shook for 1 hour, until certain amounts of immunogenic conjugates were non-covalently conjugated with NDs, resulting aggregation of the present immunogenic conjugates on the surface of NDs, thus, the present immunogenic composition was produced.

### Immunogenic Composition Characterization

The amount of immunogenic conjugates coated on NDs and the amount of free immunogenic conjugates remained in solution were determined by an ultraviolet-visible (US-Vis) spectrophotometer (U-3310, Hitachi). Absorption spectra of immunogenic conjugates with known concentrations (i.e. 100 μg/mL) were first acquired for the construction of a standard curve. The immunogenic conjugates-ND mixtures (typically with a total volume of 1 mL for each sample) were then centrifuged at 20,000×g for 5 min to precipitate NDs. The amounts of immunogenic conjugates remaining in the supernatants were quantified by interpolating their absorption strengths at 280 nm to that of the standard curve. Next, the ratio of agonists conjugated to antigens were determined by measuring absorbances at 260 nm and 280 nm using hydrophobic interaction chromatography (HIC) or an ultraviolet-visible (US-Vis) spectrophotometer (U-3310, Hitachi), depending on different agonists.

Regarding TLR5 and TLR7 agonists, the ratio of agonist-conjugated to antigen (R_{TLR5} and R_{TLR7}) were respectively calculated by the following equation (1):

$${R}_{TLR 5} or {R}_{TLR 7} = 2.77 \times {A}_{254} / {A}_{280} - ( 0.35 \times {A}_{254} ) \qquad ( 1 )$$

wherein the value 0.35 is the correction factor due to the absorbance of TLR5 or TLR7 agonists at 280 nm.

Regarding TLR9 agonist, the ratio of agonist-conjugated to antigen (R_{TLR9}) was calculated based on the following equations (2) to (3):

$${C}_{HA} \cdot {ε}_{260 nm , antigen} + {C}_{agonist} \cdot {ε}_{260 nm , agonist} = {A}_{260} \qquad ( 2 )$$

$${C}_{HA} \cdot {ε}_{280 nm , antigen} + {C}_{agonist} \cdot {ε}_{280 nm , agonist} = {A}_{280} \qquad ( 3 )$$

$${ε}_{260 nm , antigen} = 115972 {M}^{- 1} {cm}^{- 1} ; {ε}_{280 nm , antigen} = 218404 {M}^{- 1} {cm}^{- 1}$$

$${ε}_{260 nm , agonist} = 181100 {M}^{- 1} {cm}^{- 1} ; {ε}_{280 nm , agonist} = 99396 {M}^{- 1} {cm}^{- 1}$$

where C_{antigen} and C_{agonist} are molarities of antigens and agonists, respectively.

Further, hydrodynamic sizes and zeta potentials of the present immunogenic composition and antigen-coated NDs were respectively determined by using a particle size and zeta potential analyzer (DelsaNano C, Beckman Coulter).

### Animal Studies

BALB/cJ mice were purchased from BioLASCO Co., Ltd, and housed in a polycarbonate cage (cage size of 33.2 cm×21.5 cm×21 cm) at an environmentally monitored, well-ventilated room maintained at a temperature of 20-26° C. and a relative humidity of 40%-70%. Normal illumination was provided and maintaining 12-hour light/dark cycle. All mice in the study had oral intake of water and food ad libitum.

### I. Single Dose for Subcutaneous Administration

The present immunogenic composition was formulated as a water-in-oil emulsion for injection by mixing the present immunogenic composition (35 μL) with incomplete Freund's adjuvant (IFA) (65 μL). BALB/cJ mice were divided into five groups (Groups I to V, 10 mice per each group), and each mouse was subcutaneously injected with single dose of designated vaccines on day 0 (volume: 100 μL), Group I: hemagglutinin-TLR7 agonist conjugated with NDs (hereinafter, HA-TLR7-LVA), Group II: hemagglutinin-TLR7 agonist without conjugated with NDs (hereinafter, HA-TLR7-PBS), Group III: hemagglutinin-TLR9 agonist conjugated with NDs (hereinafter, HA-TLR9-LVA), Group IV: hemagglutinin-TLR9 agonist without conjugated with NDs (hereinafter, HA-TLR9-PBS), and Group V: NDs solution (hereinafter: LVA). Murine blood from Groups I to V mice were respectively collected on day 0 and day 28, and the amount of antibodies IgG and its specific subclasses (IgG1, IgG2a, and IgG2b) in sera of each mouse was determined.

### II. Subcutaneous Administration for Immunization

BALB/cJ mice were divided into five groups (Groups I to V, n=10 per each group) and subcutaneously injected with designated vaccines twice on day 0 and day 14, (total injection volume: 100 L). Group I: hemagglutinin-TLR5 agonist conjugated with NDs (hereinafter, HA-TLR5-LVA), Group II: hemagglutinin-TLR5 agonist without conjugated with NDs (hereinafter, HA-TLR5-PBS), Group III: HA-TLR7-LVA, Group IV: HA-TLR7-PBS, and Group V: LVA. Murine blood from Groups I to V mice were respectively collected on day 0 and day 35, the amount of IgG, IgG1, IgG2a, and IgG2b antibodies in sera, cytokines (e.g., IFN-γ, IL2, TNFα, CD40L, and CD107a), and cell numbers of cytotoxic and helper T cells of each mouse were determined.

### III. Subcutaneous Administration for Long-Term Immunization

Similar to subcutaneous administration, BALB/cJ mice were divided into five groups (Groups I to V, n=10 per each group) and subcutaneously injected with designated vaccines twice on day 0 and day 14, (total injection volume: 100 L). Group I: HA-TLR7-LVA, Group II: HA-TLR7-PBS, Group III: HA-TLR9-LVA, Group IV: HA-TLR9-PBS, and Group V: LVA. Murine blood from Groups I to V mice were respectively collected on day 0 and day 49, the amount of IgG, IgG1, IgG2a, and IgG2b antibodies in sera, cytokines, and cell numbers of cytotoxic and helper T cells of each mouse were determined.

### IgG Antibody Assay

Murine blood was collected from the submandibular veins of vaccinated or non-vaccinated mice. Antigen-specific IgG antibody responses of the immunized mice were evaluated by using ELISA with the collected mouse sera measured in a microplate reader (GloMax, Promega).

### Enzyme-Linked Immunospot (ELISpot) Assay

Murine blood was collected from the submandibular veins of vaccinated or non-vaccinated mice on designated days after immunization. Antibody responses of the immunized mice were evaluated and measured using ELISA assay and OVA-specific IgG, IgG1, IgG2a, and IgG2b kits (OVA sIgG; Cusabio, TX, USA) in a microplate reader (GloMax, Promega, WI, USA).

IFN-γ ELISpot assays were performed by following the manufacturers' instructions (R&D Systems). Specifically, in this experiment, freshly isolated splenocytes (1×10⁶) were plated in triplicates into 96-well ELISpot plates and stimulated for 48 h at 37° C. in an incubator with 5% CO₂. After cell removal, detection antibodies were added into plates, which were then placed at 4° C. overnight, followed by the addition of streptavidin-alkaline phosphatase for reacting further 2 hours at room temperature. A reacting agent containing nitro blue tetrazolium (NBT)/5-bromo-4-chloro-3-indolylphosphate was added subsequently into the plate for 1-hour incubation in the dark to perform spot detection. IFN-γ-specific spot-forming cells were counted using a cell analyzer (CTL S6 Universal Analyzer ImmunoSpot).

### Flow Cytometry Analysis

Cell sorting was conducted via flow cytometry with fluorochrome-conjugated antibodies. After a final wash before sorting, peripheral blood mononuclear cells (PBSCs) collected from murine blood were filtered through a 40-μm nylon cell strainer (Becton Dickinson, USA) for removal of cell clumps, and then subjected to sorting using a flow cytometer (FACSAria cell sorter, produced by Becton Dickinson). Sorted populations were collected in a complete medium (10% FBS in RPMI 1640) for further use.

### Statistics

Statistical analysis was conducted with GraphPad Prism 8 software. Two-way ANOVA with uncorrected Fisher's least significant differences test was applied for the analysis of two independent variables. A comparison between the two samples was done with the unpaired t-test. Error bars are shown as mean±SD. ns, non-significant, *p<0.05, **p<0.01, ***p<0.001, ****p<0.0001.

### Example 1 Characterization of the Present Immunogenic Composition

In this example, whether the nanodiamonds (NDs) could serve as a stable, tailorable platform for vaccination was investigated. To this purpose, the present immunogenic composition was produced according to procedures described in the “Materials and Methods” section, thereby giving rise to the immunogenic composition of “HA-TLR5-LVA”, “HA-TLR7-LVA”, and “HA-TLR9-LVA”, respectively.

The size and zeta potential of the nanodiamonds before and after loaded with immunogenic conjugates (i.e., the present agonist-linker-antigen conjugates: “HA-TLR5”, “HA-TLR7”, and “HA-TLR9”) were analyzed by using a spectrophotometer.

Next, the amounts of the present immunogenic conjugates adsorbed onto NDs, and the ratios of antigen-to-agonist conjugation were determined by ultraviolet-visible (US-Vis) spectrophotometer (U-3310, Hitachi).

### Example 2 the Present Immunogenic Composition Enhances the Immune Response

### 2.1 Single Dose for Subcutaneous Administration

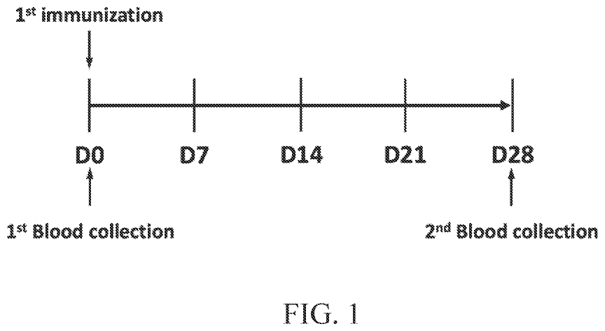

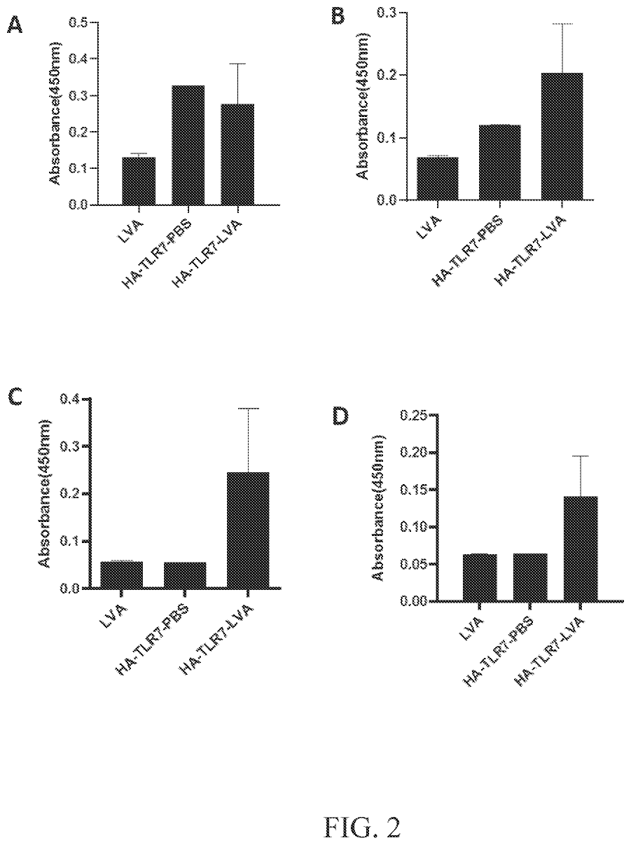

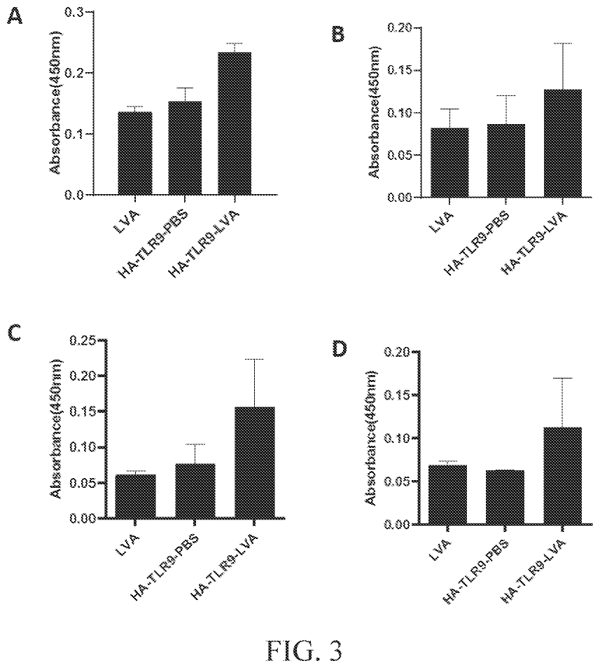

In this example, whether the present immunogenic compositions enhanced rapid humoral immune response was investigated. To this purpose, mice were randomly divided into five groups (Groups I to V, 10 for each group), each group received designated treatment in accordance with the scheme depicted in FIG. 1 and procedures described in “Materials and Methods” section. Specifically, at day 0, mice in Group I received a single dose of the HA-TLR7-LVA vaccine, and mice in Group III received a single dose of the HA-TLR9-LVA vaccine. For comparison, at day 0, mice in Group II received a single dose of the HA-TLR7-PBS vaccine, mice in Group IV received a single dose of the HA-TLR9-PBS vaccine, and mice in Group V received a single dose of the LVA vaccine. Murine blood of Groups I to V mice was collected on days 0 and 28, and the amount of IgG and its subclass antibodies after immunization were measured by serum IgG ELISA against full-length recombinant H1 protein of influenza A virus (A/California/07/2009). Results are depicted in FIGS. 2 to 3.

FIG. 2 depicts the quantitative results of the titers of IgG and its subclass antibodies after immunization of the present HA-TLR7-LVA, and comparative vaccines HA-TLR7-PBS and LVA. It was found in the humoral immune responses, that the mean endpoint titer of Group I mice on day 28 increased by 2.33±0.90 folds in total IgG (panel (A) of FIG. 2), as compared to that of the control group LVA (Group V). Further, it was found that the mean endpoint titers of three IgG subclasses IgG1, IgG2a, and IgG2b all increased by 3.04±0.75, 4.90±1.20, and 2.33±0.05 folds, respectively (FIG. 2, panel (B)-(D)).

FIG. 3 depicts the quantitative results of the titers of IgG and its subclass antibodies after immunization of the present HA-TLR9-LVA and comparative vaccines HA-TLR9-PBS and LVA. It was found in the treatment of the present immunogenic composition, the mean endpoint titer of Group III mice on day 28 increased by 1.73±0.12 and 1.65±0.18 folds in total IgG (panel (A) of FIG. 3), as compared to that of the control groups LVA (Group V) and HA-TLR9-PBS (without nanodiamonds, Group IV). Further, it was found that the mean endpoint titers of three IgG subclasses IgG1, IgG2a, and IgG2b all increased by 1.65±0.05, 2.67±0.06, and 1.82±0.05 folds, respectively (FIG. 3, panel (B)-(D)).

The results depicted in FIGS. 2 to 3 collectively indicated the present immunogenic composition elicits specific antibodies with Fc-effector function activity, therefore can enhance superior immune response.

### 2.2 Subcutaneous Administration for Immunization

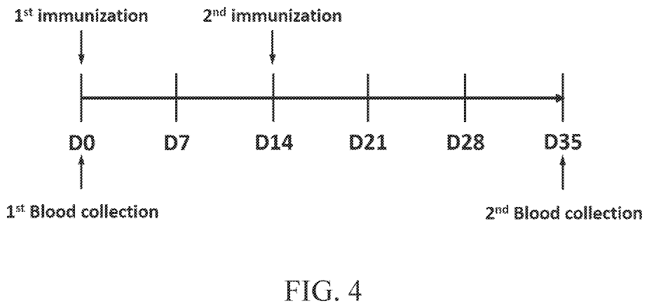

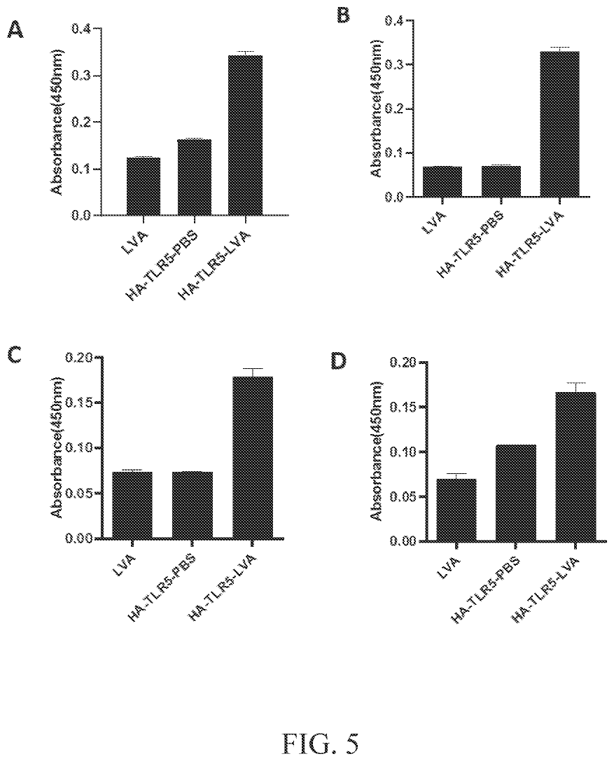

In this example, the immunization effects of the present immunogenic composition on humoral and cell-mediated immunities were investigated. To this purpose, mice were divided into five groups (Groups I to V), and each mouse in Group I and Group III was subcutaneously injected with double vaccine of the present immunogenic compositions “HA-TLR5-LVA” and “HA-TLR7-LVA” on day 0 and day 14 in accordance with the scheme depicted in FIG. 4 and “Materials and Methods” section. For comparison, mice in Group II, Group IV, and Group V respectively received HA-TLR5-PBS, HA-TLR7-PBS, and LVA on day 0 and day 14. Murine blood from Groups I to V mice were respectively collected on day 0 and day 35. Humoral immune responses of Groups I, II and V mice (i.e., HA-TLR5-LVA, HA-TLR5-PBS, and LVA immunization) were measured by serum IgG ELISA against full-length recombinant H1 protein of influenza A viruses (A/California/07/2009). Further, expression of intracellular cytokines for cytotoxic and helper T cells derived from all five groups of mice was determined by flow cytometry. Results are depicted in FIGS. 5 to 7.

The quantitative results of humoral immune responses are depicted in FIG. 5. It was found that, compared to control groups (LVA and HA-TRL5-PBS), the mean endpoint IgG titer of HA-TLR5-LVA group on day 35 increased 2.83±0.07 and 2.50±0.06 folds after twice immunization (FIG. 5, panel (A)). It was also found that the mean endpoint titers of three IgG subclasses IgG1, IgG2a, and IgG2b were all increased by 4.13±0.02, 2.50±0.01, and 2.48±0.02 folds, as depicted in panels (B)-(D) of FIG. 5.

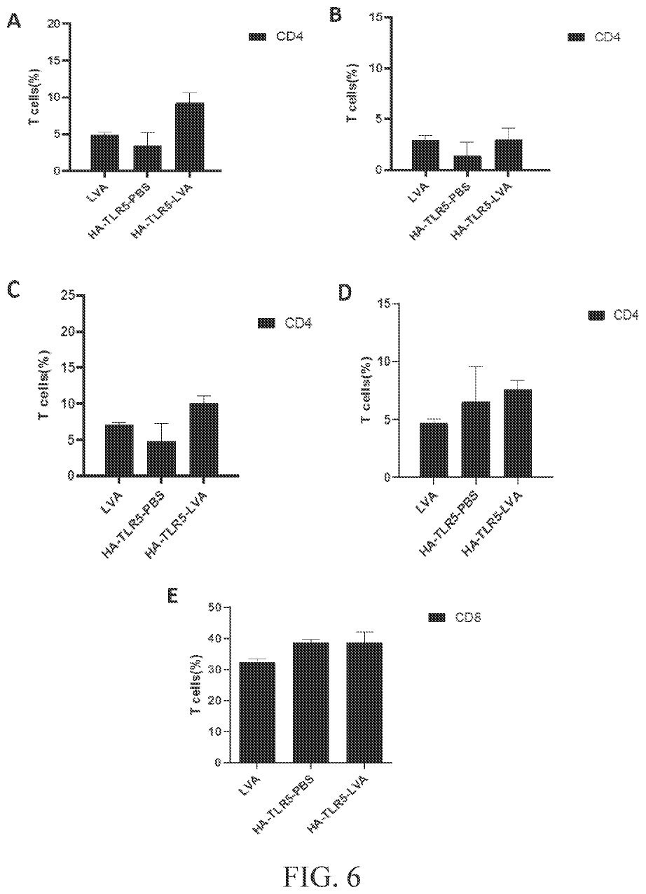

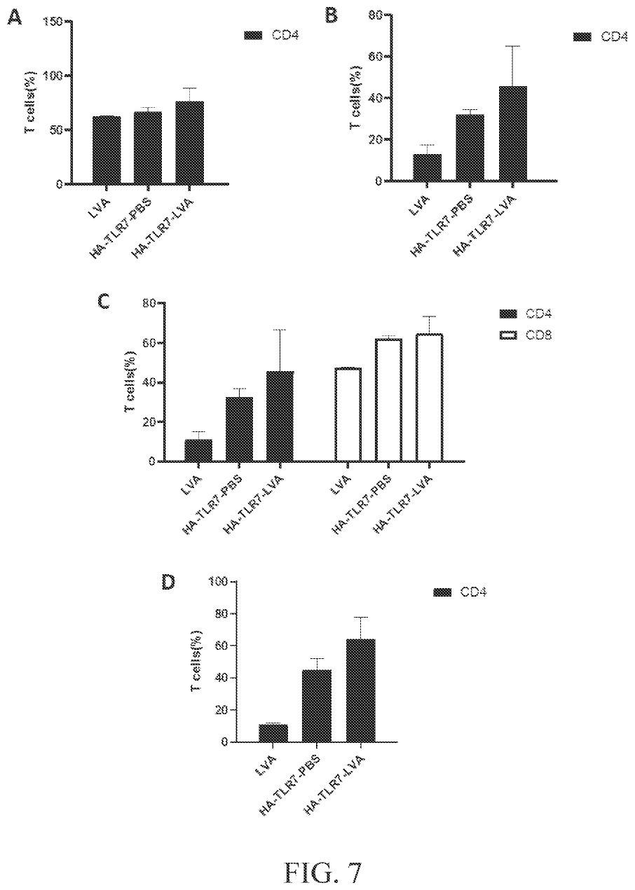

As for cell-mediated immunity, it was found that compared to the control group mice received HA-TLR5-PBS, the amount of CD4⁺ T cells that expressed pro-inflammatory cytokines IFN-γ, interleukin-2 (IL-2), and tumor necrosis factor-α (TNF-α) of double-dose immunized mice (Group I) were significantly increased (FIG. 6, panels (A) to (C)). It was also found in subcutaneous administration of the present HA-TLR7-LVA that, compared to control groups, both CD4⁺ and CD8⁺ cells were significantly activated, in which the expression of cytokines IL-2, TNF-α, and CD40L in CD4⁺ cells was increased by 2.80±0.90, 4.00±0.85, and 6.30±0.75 folds, respectively (FIG. 7, panels (B) to (D)), and the expression of TNF-α in CD8⁺ cells was increased by 1.4±0.41 folds (FIG. 7, panel (C)).

The data depicted in FIGS. 5 to 7 collectively indicated that the present immunogenic composition possesses superior immunization effects on humoral and cell-mediated immunities.

### 2.3 Subcutaneous Administration for Long-Term Immunization

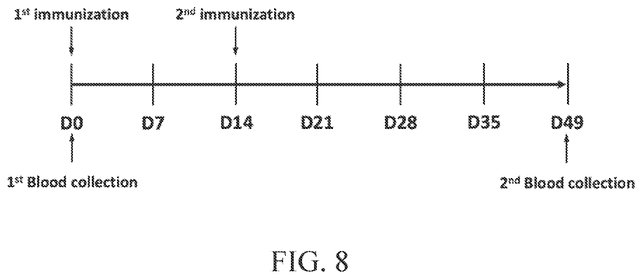

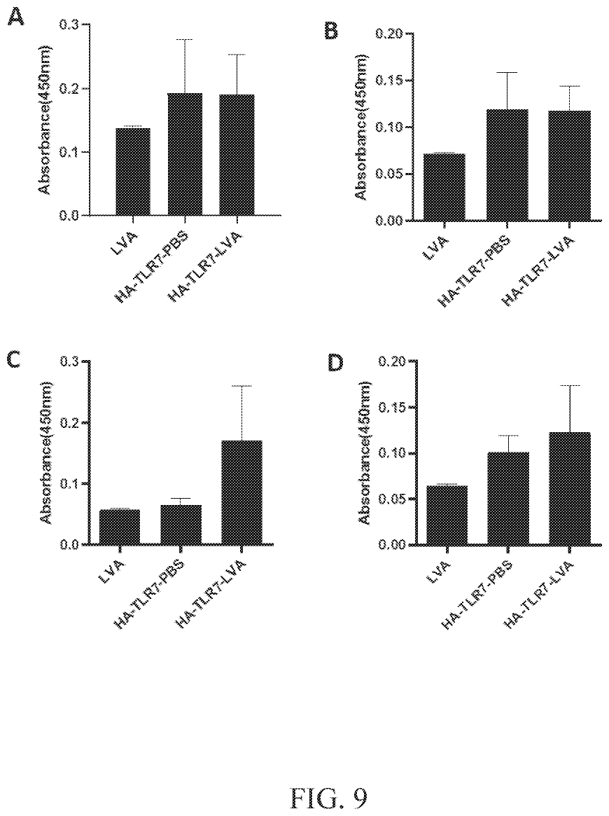

In this example, the long-term immunization effects of the present immunogenic composition on humoral and cell-mediated immunities were investigated. To this purpose, mice were divided into five groups (Groups I to V), in which each mouse of Group I and Group III was subcutaneously injected with double-doses of the present immunogenic compositions “HA-TLR7-LVA” and “HA-TLR9-LVA” on day 0 and day 14 in accordance with the scheme depicted in FIG. 8 and “Materials and Methods” section. For comparison, mice in Group II, Group IV, and Group V respectively received HA-TLR7-PBS, HA-TLR9-PBS, and LVA on day 0 and day 14. Murine blood from Groups I to V mice were respectively collected on day 0 and day 49. Humoral immune responses were measured by serum IgG ELISA against full-length recombinant H1 protein of influenza A viruses (A/California/07/2009), and expression of intracellular cytokines for cytotoxic and helper T cells derived from all five groups of mice was determined by flow cytometry. Results are depicted in FIGS. 9 to 12.

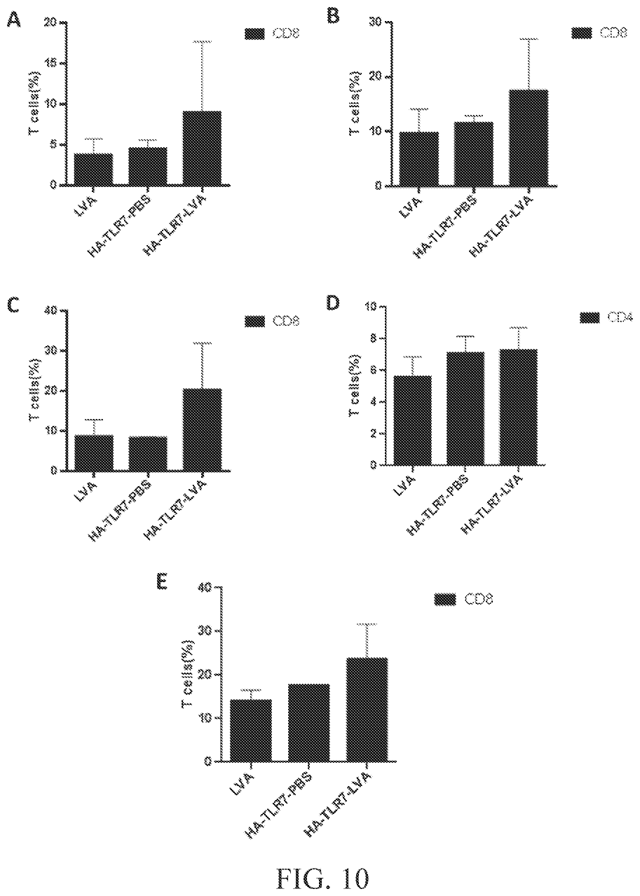

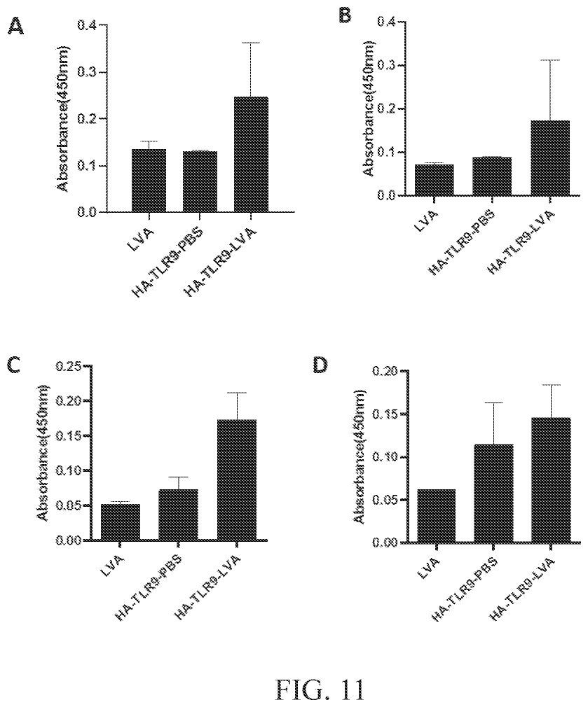

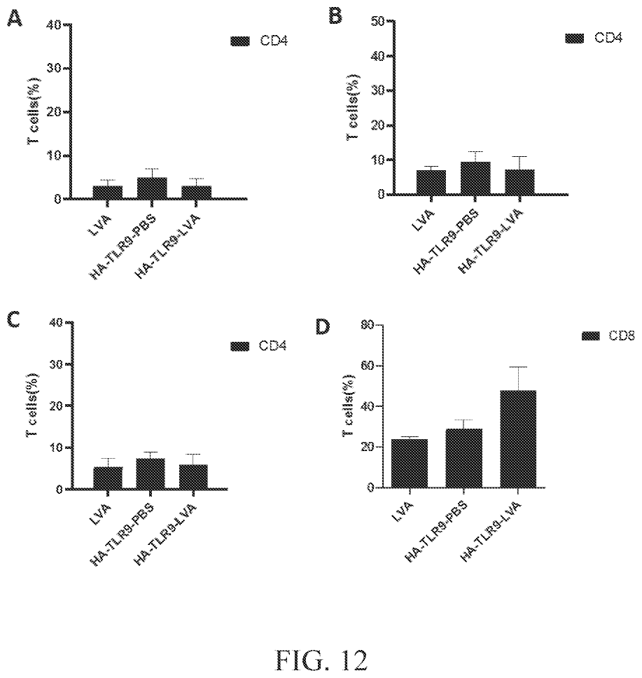

It was found that, compared to control groups, after double immunization, the numbers of antibodies for both present immunogenic compositions “HA-TLR7-LVA” and “HA-TLR9-LVA” significantly increased after a prolonged time (longer than 1.5 months) (FIGS. 9 and 11). In addition, it was also found that both cytotoxic and helper T cells immunized by the present immunogenic compositions remain activated even after 45 days (FIGS. 10 and 12).

The results depicted in FIGS. 9 to 12 collectively indicate that the present immunogenic composition possesses merits of long-term immunization effects on humoral and cell-mediated immunities.

Taken together, the present immunogenic composition can effectively enhance immunogenicity and, therefore possess superior potential for vaccination and medication.

It will be understood that the above description of embodiments is given by way of example only and that various modifications may be made by those with ordinary skills in the art. The above specification, examples, and data provide a complete description of the structure and use of exemplary embodiments of the invention. Although various embodiments of the invention have been described above with a certain degree of particularity, or with reference to one or more individual embodiments, those with ordinary skills in the art could make numerous alterations to the disclosed embodiments without departing from the spirit or scope of this invention.

What is claimed is:

1. An immunogenic composition, comprising:
a nanodiamond; and
an immunogenic conjugate of formula (I): X-L-Y, wherein X is an agonist module, L is a linker unit, and Y is an antigen,
wherein,
the immunogenic conjugate is coupled to the outer surface of the nanodiamond.

2. The immunogenic composition of claim 1, wherein the nanodiamond is about 1 to 1,000 nm in diameter.

3. The immunogenic composition of claim 1, wherein the agonist module is an agonist selected from the group consisting of a Toll-like receptor (TLR) agonist, a retinoic acid-inducible gene I (RIG-I)-like receptor agonist, a C-type lectin receptor (CLR) agonist, a NOD-like receptor (NLR) agonist, a stimulator of interferon genes (STING) agonist, a pattern recognition receptor (PRR) agonist, and a combination thereof.

4. The immunogenic composition of claim 3, wherein the TLR agonist is selected from the group consisting of a TLR5 agonist, a TLR7 agonist, and a TLR9 agonist.

5. The immunogenic composition of claim 4, wherein the TLR5 agonist is flagellin.

6. The immunogenic composition of claim 4, wherein the TLR7 agonist is 1H-imidazo[4,5-C]quinoline-1-propanamine,4-amino-2-(ethoxymethyl).

7. The immunogenic composition of claim 4, wherein the TLR9 agonist is cytosine-phosphate-guanine oligonucleotide (ODN) 2006 having a substituted amino group at its 5′-end.

8. The immunogenic composition of claim 1, wherein the linker unit is a succinimide derivative or a PEG-based compound.

9. The immunogenic composition of claim 8, wherein the succinimide derivative is N-(ε-maleimidocaproyloxy) succinimide ester or N-γ-maleimidobutyryl-oxysuccinimide ester.

10. The immunogenic composition of claim 8, wherein the PEG-based compound is Azido-PEG4-NHS ester.

11. The immunogenic composition of claim 1, wherein the antigen is derived from an autogenous, allogeneic, heterophile, or xenogeneic source.

12. The immunogenic composition of claim 11, wherein the antigen is an autoantigen.

13. The immunogenic composition of claim 12, wherein the autoantigen is derived from a protein selected from the group consisting of proinsulin, myelin basic protein (MBP), insulin, aluminum-formulated glutamic acid decarboxylase (GAD-alum), amyloid precursor protein (APP), heat shock protein, major histocompatibility complex (MHC), human leukocyte antigen (HLA), myelin oligodendrocyte glycoprotein (MOG), and ovalbumin.

14. The immunogenic composition of claim 11, wherein the antigen is a tumor antigen, a bacterial antigen, a viral antigen, a fungal antigen, a parasitic antigen, or a combination thereof.

15. The immunogenic composition of claim 14, wherein the tumor antigen is selected from the group consisting of neoantigen, tumor-derived lysate, alpha-fetoprotein (AFP), carcinoembryonic antigen (CEA), mucin, epithelial tumor antigen (ETA), tyrosinase, melanoma-associated antigen (MAGE), RAS protein, and tumor suppressor protein.

16. The immunogenic composition of claim 14, wherein the bacterial antigen is derived from a bacterial species selected from the group consisting of Actinomyces, Aeromonas, Arthrobacter, Bacillus, Bacteroides, Bordetella, Borrelia, Brucella, Campylobacter, Chlamydia, Citrobacter, Clostridium, Corynebacterium, Escherichia, Enterobacter, Gardnerella, Helicobacter, Haemophilus, Klebsiella, Legionella, Listeria, Mycobacterium, Neisseria, Nocardia, Pasteurella, Proteus, Pseudomonas, Ureaplasma, Salmonella, Shigella, Spirillum, Spirochaeta, Staphylococcus, Streptobacillus, Streptococcus, Streptomyces, Treponema, and Yersinia.

17. The immunogenic composition of claim 14, wherein the viral antigen is derived from a viral species selected from the group consisting of Adenovirus, Alphacoronavirus, Betacoronavirus, Cytomegalovirus, Deltainfluenzavirus, Deltacoronavirus, Gammacoronavirus, Hepatovirus, Influenza A virus, Influenza B virus, Influenza C virus, Influenza D virus, Lentivirus, Letovirus, Lymphocryptovirus, Orthopneumovirus, Orthopoxvirus, Papillomavirus, Quaranjavirus, Rotavirus, Simplexvirus, and Varicellovirus.

18. The immunogenic composition of claim 17, wherein the viral antigen is a hemagglutinin of the Influenza A virus.

19. The immunogenic composition of claim 14, wherein the fungal antigen is derived from a fungal species that causes a fungal infection selected from the group consisting of aspergillosis, blastomycosis, candidiasis, chromoblastomycosis, cryptococcosis, histoplasmosis, mycetoma, paracoccidioidomycosis, ringworm, and tinea versicolor.

20. The immunogenic composition of claim 14, wherein the parasitic antigen is derived from a parasite species that causes a parasitic infection selected from the group consisting of African trypanosomiasis, amebiasis, Chagas disease, echinococcosis, fascioliasis, hookworm disease, hymenolepis, leishmaniasis, neurocysticercosis, onchocerciasis, Plasmodium infection, paragonimiasis, Pneumocystis pneumonia (PCP), schistosomiasis, trichomoniasis, taeniasis, and trichuriasis.

21. A method for preventing or treating a disease in a subject in need thereof comprising administering an effective amount of the immunogenic composition of claim 1 to the subject.

22. The method of claim 21, wherein the disease is a cancer or an infectious disease.

23. The method of claim 22, wherein the cancer is selected from the group consisting of bladder cancer, bone cancer, bone marrow cancer, brain cancer, breast cancer, cholangiocarcinoma, colon cancer, esophagus cancer, gastrointestinal cancer, gum cancer, head and neck cancer, kidney cancer, liver cancer, lung cancer, nasopharyngeal carcinoma, leukemia, lymphoma, ovary cancer, prostate cancer, skin cancer, stomach cancer, testis cancer, tongue cancer, and uterus cancer.

24. The method of claim 22, wherein the infectious disease is caused by a pathogen selected from the group consisting of a bacterium, virus, fungus, parasite, and a combination thereof.

25. The method of claim 24, wherein the infectious disease is caused by Influenza A virus, Influenza B virus, Influenza C virus, Influenza D virus, or severe acute respiratory syndrome-related coronavirus (SARS-CoV-2).

26. The method of claim 21, wherein the subject is a mammal.

27. The method of claim 26, wherein the mammal is a human.
